(19) United States
(12) Patent Application Publication
(10) Pub. No.: US 2010/0069790 A1
(43) Pub. Date: Mar. 18, 2010

(54) MEDICAL APPARATUS WITH CANNULA AND RELEASABLE HANDLE ASSEMBLY FOR ACCESSING REMOTE ANATOMICAL SITES

(76) Inventor: Scott Green, Davison, MI (US)

Correspondence Address: HOWARD & HOWARD ATTORNEYS PLLC, 450 West Fourth Street, Royal Oak, MI, 48067, US

(21) Appl. No.: 12/623,208

(22) Filed: Nov. 20, 2009

Related U.S. Application Data
(63) Continuation of application No. 11/428,999, filed on Jul. 6, 2006, now abandoned.
(60) Provisional application No. 60/697,311, filed on Jul. 7, 2005.

(51) Int. Cl. A61B 10/02; A61M 5/00

(52) U.S. Cl. 600/567; 604/165.01

(57) ABSTRACT

A medical apparatus (29) for accessing a remote anatomical site is provided. The medical apparatus (29) includes a handle assembly (33) for releasably retaining a cannula (31). The handle assembly (33) includes a collar (40) slidable along a body portion (37) between a lock position for holding catches (36) on flexible detent arms (34) in an inward position and in engagement with a recess (30) in the cannula (31) to retain the cannula (31), and a release position for allowing the catches (36) to move radially outwardly to release the cannula (31). A channel (28) defined in the body portion (37) contains keys (68) that allow for insertion of the cannula (31) when aligned with grooves (64) defined in an outer surface of the cannula (31). A pair of grip tabs (48) extend radially in opposite directions from the collar (40) for manually moving the collar (40) to the release position and a pair of flexible arms (44), acting as springs, extend in opposite directions from the grip tabs (48) in outwardly bowed sections to bias the collar (40) to the lock position to retain the cannula (31) to the handle assembly (33).

### RELATED APPLICATIONS

The application is a Continuation Application of U.S. patent application Ser. No. 11/428,999, filed Jul. 6, 2006, which claims the benefit of U.S. Provisional Patent Application No. 60/697,311, filed Jul. 7, 2005, the advantages and disclosures of both of which are hereby incorporated by reference.

### FIELD OF THE INVENTION

The present invention relates to a medical apparatus for penetrating tissue to access a remote anatomical site. More specifically, the present invention relates to the medical apparatus having a cannula and a releasable handle assembly for releasably retaining a proximal end of the cannula to perform biopsies and otherwise access remote anatomical sites.

### BACKGROUND OF THE INVENTION

It is well known in the art to provide a medical apparatus having a releasable handle assembly for releasably retaining a medical device such as a cannula for performing biopsies or otherwise accessing remote anatomical sites. Furthermore, various releasable handle assemblies for supporting the proximal end of the cannula are well known to include a handle portion for manually grasping the handle assembly and a body portion supported by the handle portion and defining a channel for receiving the proximal end of the cannula. Examples of such devices are disclosed in the U.S. Pat. Nos. 6,340,351 to Goldenberg and 7,001,342 to Faciszewski.

In the '351 patent to Goldenberg, the medical apparatus includes a handle assembly having a body portion with detent pockets. A hub is rigidly fixed to an outer cannula and the hub includes a plurality of detent arms that are designed to snap-lock into the detent pockets of the body portion when inserting the hub into the body portion. The outer cannula can then be removed from the handle assembly by pressing the detent arms inwardly to release them from their associated detent pockets. In the '351 patent, the hub fixed to the outer cannula obstructs another cannula from being slid over the outer cannula.

In the '342 patent to Faciszewski, a handle assembly is removably coupled to a cannula by means of a lock nut being threaded onto a threaded split-collar of the handle assembly to lock the handle assembly to the cannula. The cannula in the '342 patent can be released by unthreading the lock nut from the threaded split-collar of the handle assembly and sliding the threaded split-collar and the lock nut off of the cannula.

While there are numerous releasable handle assemblies for releasably retaining medical devices such as a cannula and other remote access devices, there remains an opportunity for a medical apparatus with a releasable handle assembly that provides rigid support of the cannula while allowing simpler and faster coupling and decoupling of the cannula.

### SUMMARY OF THE INVENTION AND ADVANTAGES

The present invention provides a medical apparatus having a cannula for accessing remote anatomical sites and a handle assembly for supporting a proximal end of the cannula. The cannula includes a locking feature adjacent the proximal end. The handle assembly includes a handle portion for manually grasping the handle assembly, and a body portion supported by the handle portion and defining a channel for receiving the proximal end of the cannula. The body portion presents a detent moveable between an inward position extending into the channel for engaging the locking feature to retain the cannula and an outward position for disengaging from the locking feature to allow the cannula to move in and out of the body portion. A component is slidable along the body portion between a lock position for holding the detent in the inward position to retain the cannula and a release position for allowing the detent to move to the outward position to release the cannula.

In another aspect of the present invention, a second cannula is provided and sized for sliding over the other cannula and the locking feature.

Although systems utilizing a cannula with a releasable handle assembly have been employed in a variety of ways, the present invention allows for rigid support of the cannula, while at the same time permitting simple and rapid coupling and decoupling of the cannula.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is a perspective view of a medical apparatus of the present invention;

FIG. 2 is perspective view of a biopsy cannula and stylet which is supported and retained by a handle assembly of the present invention;

FIG. 2A is a cross-sectional view of a tip of the biopsy cannula;

FIG. 3 is an exploded perspective view of the handle assembly of the present invention;

FIG. 4A is a cross-sectional view of the handle assembly of FIG. 1 taken generally along the line 4A-4A in FIG. 1 illustrating a channel in the body portion with the biopsy cannula and stylet inserted therein and with a component of the handle assembly in a lock position to retain the biopsy cannula and stylet in the handle assembly;

FIG. 4B is a view identical to FIG. 4A except that the component of the handle assembly has been moved to a release position and the biopsy cannula and stylet are shown being removed from the handle assembly;

FIG. 5 is a cross-sectional view of the handle assembly of FIG. 1 taken generally along the line 5-5 in FIG. 1 further illustrating the channel in the body portion and the biopsy cannula and stylet inserted therein;

FIG. 6 is a cross-sectional view of the stylet of FIG. 2 taken generally along the line 6-6 in FIG. 2;

FIG. 7 is a cross-sectional view of the biopsy cannula of FIG. 2 taken generally along the line 7-7 in FIG. 2;

FIG. 8 is a cross-sectional view of a body portion of the handle assembly of FIG. 3 taken generally along the line 8-8 in FIG. 3;

FIG. 9 is a perspective view of a biopsy kit comprising the handle assembly, the biopsy cannula and stylet, an access cannula, an obturator, and an optional guide wire;

FIG. 10 is a perspective view of cement delivery tools used with the biopsy kit of FIG. 9;

FIG. 11 illustrates using the medical apparatus for penetrating tissue, e.g., cancellous bone, to access inside a vertebral body;

FIG. 12 illustrates using the medical apparatus with the guide wire;

FIG. 13 illustrates removing the handle assembly from the biopsy cannula and stylet after they have penetrated tissue;

FIG. 14 illustrates removing the stylet from the biopsy cannula;

FIG. 15A illustrates inserting the access cannula over the biopsy cannula to access the vertebral body with a distal end of the access cannula;

FIG. 15B illustrates markings on the biopsy cannula to determine a depth of insertion of the access cannula relative to the biopsy cannula;

FIG. 16 illustrates reattaching the handle assembly to the biopsy cannula;

FIG. 17 illustrates obtaining a biopsy specimen with the biopsy cannula;

FIG. 18 illustrates inserting cement delivery tools into the access cannula to deliver bone cement into the vertebral body after the biopsy specimen has been taken and the biopsy cannula has been removed;

FIG. 19 is a perspective view of an alternative medical apparatus of the present invention illustrating insertion of the alternative medical apparatus into tissue to access a remote anatomical site;

FIG. 20 illustrates removing an alternative stylet of the alternative medical apparatus from an alternative biopsy cannula of the alternative medical apparatus;

FIG. 21 illustrates removing an alternative handle assembly of the alternative medical apparatus from a hub of the alternative stylet;

FIG. 22 illustrates removing a removable hub from the alternative biopsy cannula;

FIG. 23 illustrates attaching the alternative handle assembly to a hub of an alternative access cannula;

FIG. 24 illustrate sliding the alternative access cannula over the alternative biopsy cannula to access the remote anatomical site;

FIG. 25 illustrates reattaching the removable biopsy cannula hub to the alternative biopsy cannula;

FIG. 26 illustrates reattaching the alternative handle assembly to the removable biopsy cannula hub; and

FIG. 27 illustrates obtaining a biopsy specimen with the alternative biopsy cannula.

### DETAILED DESCRIPTION OF THE INVENTION

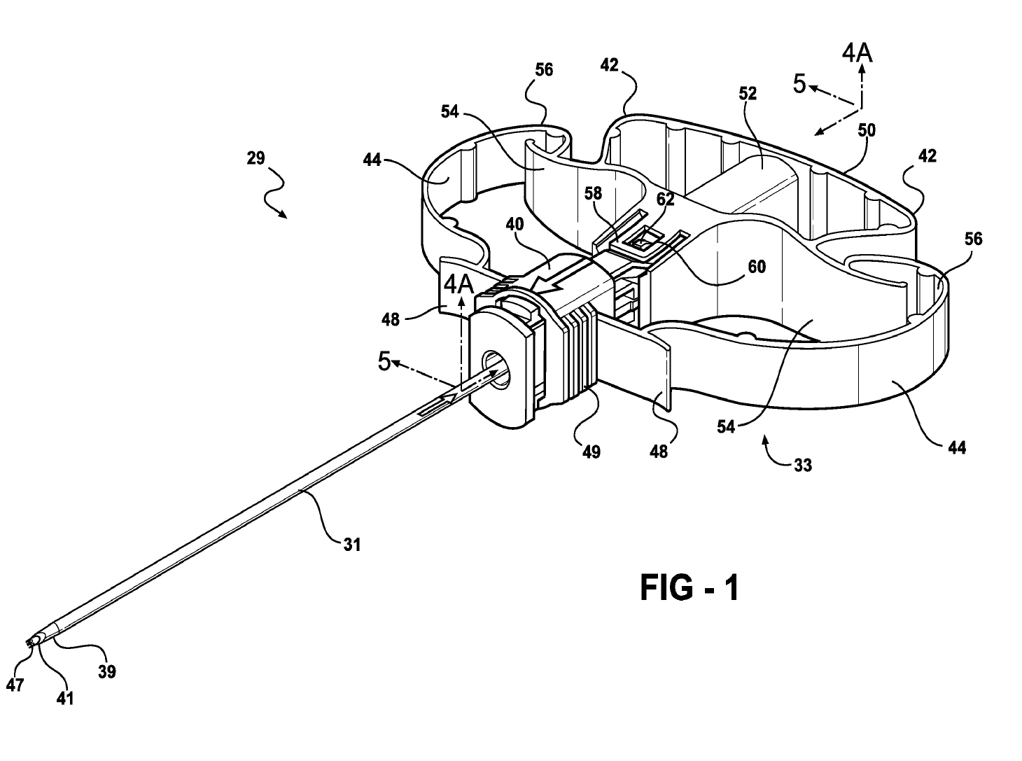

Referring to the Figures, wherein like numerals indicate like or corresponding parts throughout the several views, a medical apparatus is shown generally at 29 in FIG. 1. The medical apparatus is preferably a disposable, single-use item for penetrating tissue to access remote anatomical sites for purposes of obtaining biopsy specimens and/or other procedures such as, but not limited to, delivering materials to the remote anatomical site. Materials delivered to the remote anatomical site may include materials capable of setting to a hardened condition such as PMMA bone cement, or other materials such as medications, saline, and the like.

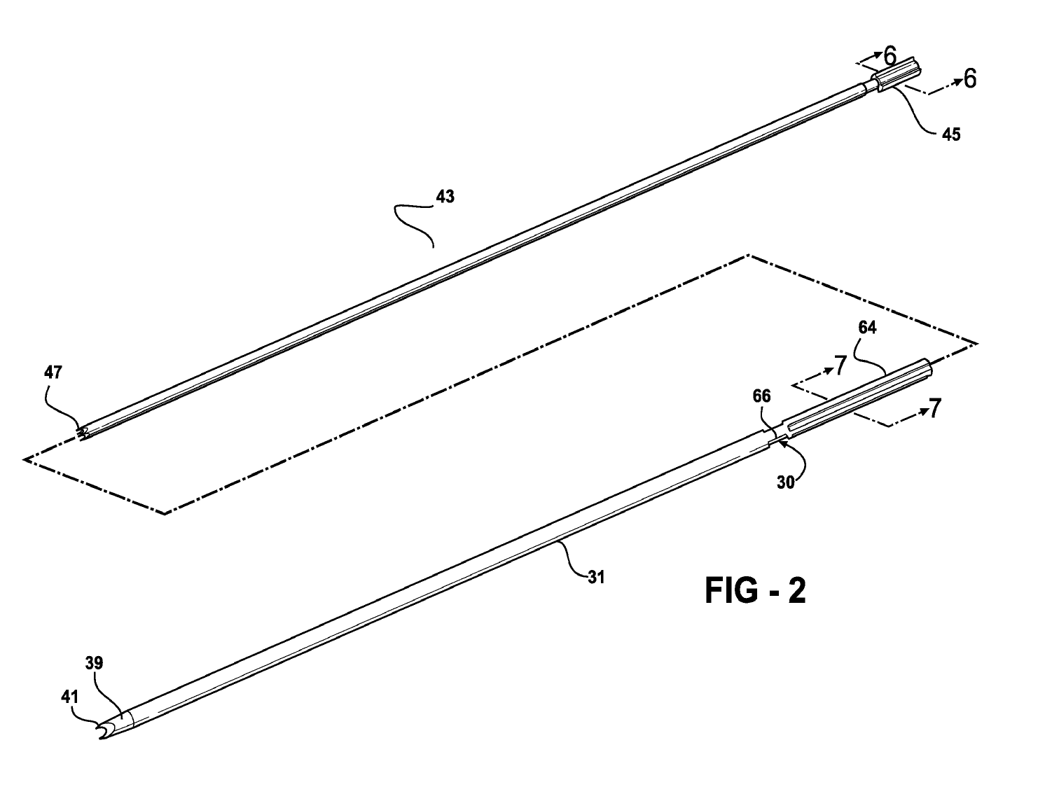

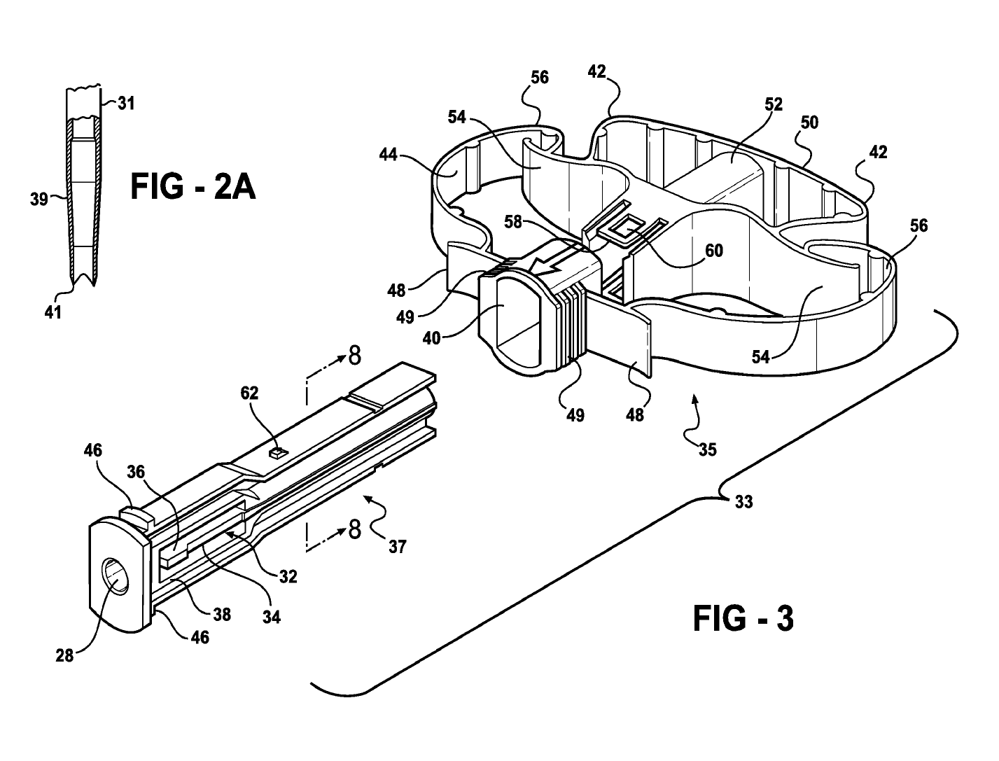

Referring to FIGS. 1 and 2, the medical apparatus 29 comprises a medical device 31 for accessing the remote anatomical site and a handle assembly 33 supporting a proximal end of the medical device 31. The medical device 31 may be a cannula, a needle, a trocar, and the like. In the preferred embodiment, the medical device 31 is a biopsy cannula 31 extending from the proximal end to a distal end. The biopsy cannula 31 is preferably a tube having a swaged portion 39 at the distal end for retaining a biopsy specimen and a sharpened, beveled tip 41 to pierce tissue (see FIG. 2A). The biopsy cannula 31 preferably has an outer diameter of 8 gauge or smaller (e.g., 10 gauge, 12 gauge, etc.) and more preferably 10 gauge or smaller.

Referring specifically to FIG. 2, a stylet 43 may be used with the biopsy cannula 31 in a known manner to penetrate tissue to access the remote anatomical site. The stylet 43 preferably includes a proximal end having a plastic hub 45 fixed thereto. The plastic hub 45 may be fixed by adhesive, UV curable materials, and the like, and may include keys/keyways or other features for preventing axial movement of the plastic hub 45. The plastic hub 45 may be formed of polycarbonate or other suitable biocompatible material. The stylet 43 extends from the proximal end to a sharpened distal end 47 for penetrating tissue. The stylet 43 may be a solid rod, or may be cannulated to receive a guide wire 122 to guide the stylet 43 and biopsy cannula 31 to the remote anatomical site using methods known to those skilled in the art. Both the biopsy cannula 31 and stylet 43 may be formed of stainless steel, e.g., 304 stainless steel, or any other suitable, biocompatible material(s). The stylet preferably has an outer diameter of 10 gauge or smaller, more preferable 12 gauge or smaller, and most preferably 14 gauge or smaller. In any event, the stylet 43 is sized for insertion into the biopsy cannula 31 with minimal spacing between the stylet 43 and an inner surface of the biopsy cannula 31.

Referring to FIG. 3, the handle assembly 33 includes a generally indicated handle portion 35, for manually grasping the handle assembly 33 and for supporting a generally indicated body portion 37. The body portion 37 defines a channel 28 for receiving the proximal end of the biopsy cannula 31, which includes a recess 30 adjacent the proximal end, as shown in FIG. 2. The recess 30 preferably includes a pair of flats 66 on opposite sides of the biopsy cannula 31 (only one side shown in FIG. 2).

The body portion 37 presents a detent 32 moveable between an inward, or a neutral, position extending into the channel 28 for retaining the biopsy cannula 31 and an outward position for allowing the biopsy cannula 31 to move in and out of the body portion 37. More specifically, the detent 32 comprises a pair of detent arms 34 integral with the body portion 37 and extending to distal ends with a catch 36 extending laterally, or medially, from each of the distal ends for engaging the recess 30 to retain the biopsy cannula 31 in the channel 28. The channel 28 is defined by a bore extending axially into the body portion 37 and the body portion 37 defines side openings 38 extending radially into the bore with the detent arms 34 being disposed in the side openings 38. The detent arms 34 and the catches 36 defining the detent 32 are inherently or normally biased to move to the radially inward position.

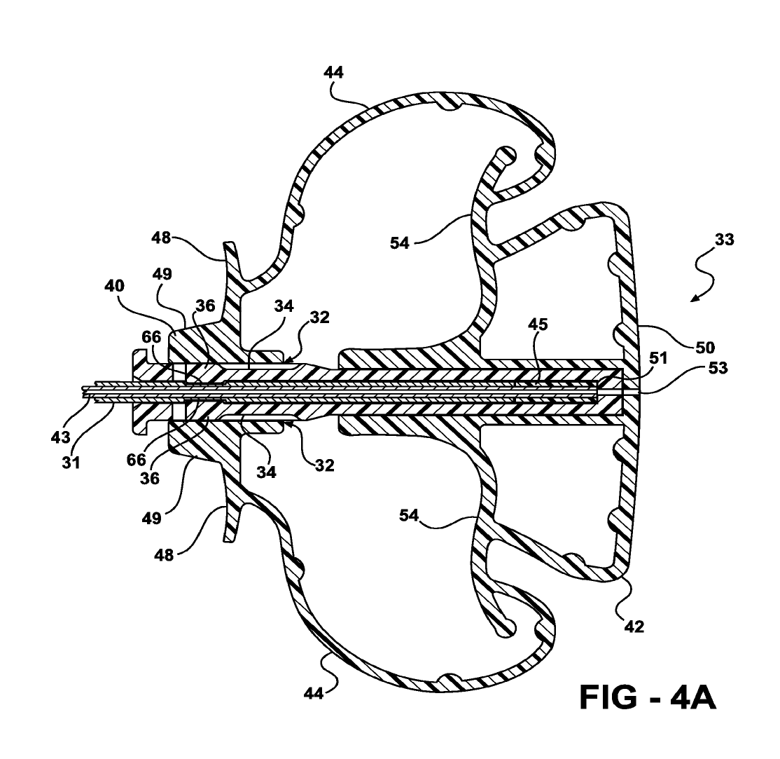

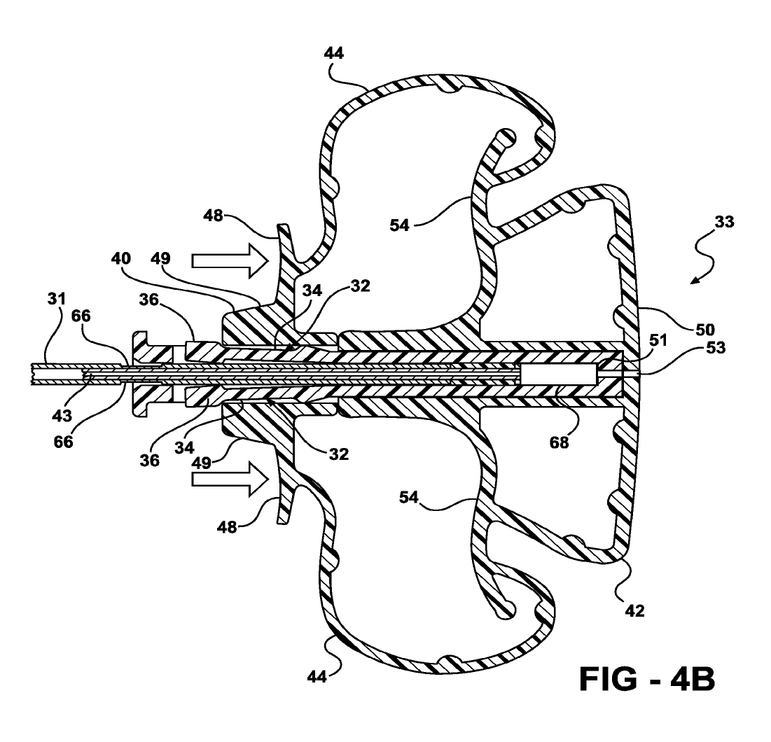

The handle assembly 33 further includes a component or collar 40 movable along the body portion 37 between a lock position for holding the detent 32 catches 36 in the inward position and a release position for allowing the detent 32 catches 36 to move to the outward position. The collar 40 is tubular and surrounds the body portion 37 for sliding along the body portion 37. FIGS. 4A and 4B show the handle assembly 33 with the collar 40 in the lock position (FIG. 4A) and in the release position (FIG. 4B). FIG. 4B further illustrates the biopsy cannula 31 being removed from the handle assembly 33. In other embodiments, not shown, the collar 40 may rotate relative to the body portion 37 between the lock position and the release position.

The handle assembly 33 also includes a part comprising a spring to urge and hold the collar 40 in the lock position and to allow the collar 40 to be moved along the body portion 37 to the release position. The handle portion 35 includes a grasping portion 42 for manually grasping and supporting the handle assembly 33 and the spring includes a pair of flexible arms 44 extending in a bendable path to interconnect the grasping portion 42 and the collar 40 for urging the collar 40 to the lock position and for bending in response to a manual force to allow the collar 40 to be moved along the body portion 37 to the release position.

A pair of stops 46 on the body portion 37 limit movement of the collar 40 in the lock position by reacting against the spring defined by the flexible arms 44. Although the spring comprises the flexible arms 44 in the embodiment illustrated, it will be understood that other alternatives are possible, such as a coil spring surrounding the body portion 37 to react with the collar 40.

A pair of grip tabs 48 extend radially in opposite directions from the collar 40 and the flexible arms 44 extend in opposite directions from the tabs 48 in outwardly bowed sections. The tabs 48 extend from the collar 40 for manually moving the collar 40 along the body portion 37 by grasping with two fingers, one for each tab 48. Ribs 49 are disposed at the intersection of the collar 40 and tabs 48 to reinforce the collar 40.

The grasping portion 42 includes a curved palm band 50 and center post 52 extending from the palm band 50 to support the body portion 37. A pair of finger grips 54 extend radially from the post 52 and are spaced between the palm band 50 and the body portion 37. The flexible arms 44 extend from the bowed sections to reverse bend sections 56 looped around and into the finger grips 54.

The body portion 37 and the center post 52 are formed separately and a mechanical connection interconnects the body portion 37 and the center post 52. More specifically, the mechanical connection includes a pair of axially extending and flexible flaps 58 presented by the post 52 and defining apertures 60 therein. Oppositely disposed lugs 62 extend radially from the body portion 37 to be disposed in the apertures 60 to retain the body portion 37 to the post 52. The flaps 58 flex outwardly as the body portion 37 is inserted into the post 52 to allow the lugs 62 to pass into the apertures 60 whereby the flaps 58 snap back to surround and engage the lugs 62.

The handle portion 35 and the flexible arms 44 and the collar 40 preferably comprise an integral organic polymeric material. Normally, the body portion 37 would also comprise the same organic polymeric material. The body portion 37 could also be integrally formed with the handle portion 35. A suitable material for the handle portion 35 and the body portion is polycarbonate (Lexan®). Of course, other biocompatible materials could also be used.

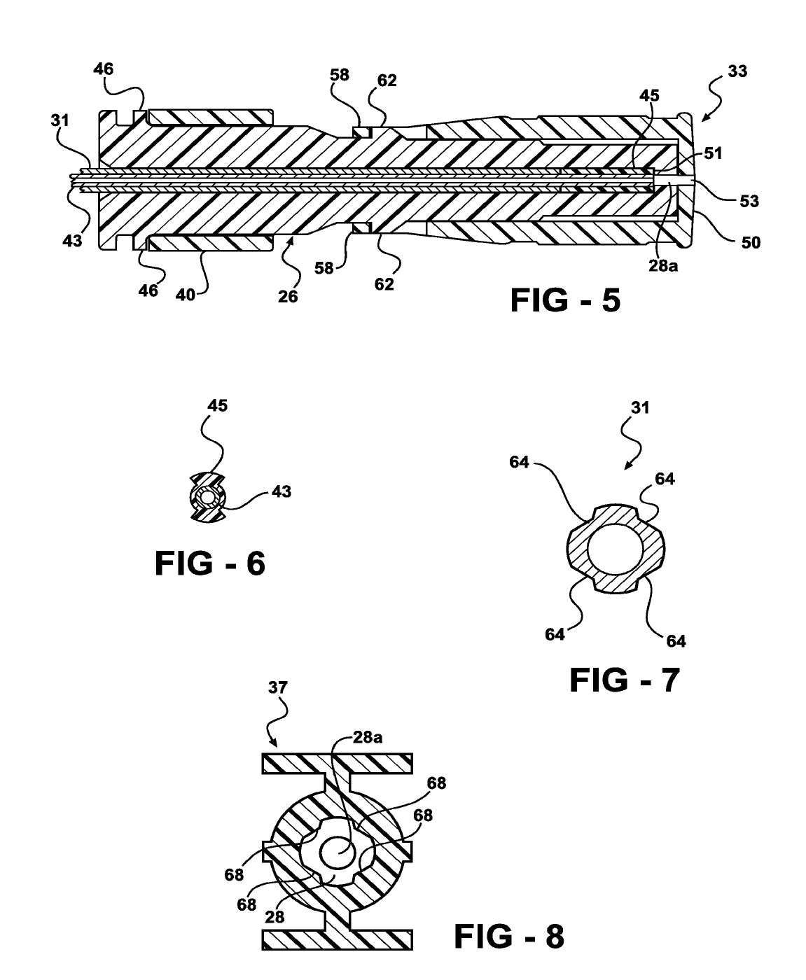

Referring to FIGS. 4A, 4B, and 5, the channel 28 extends entirely through the body portion 37 to receive the medical device, e.g., biopsy cannula 31 with stylet 43. However, the channel 28 is preferably narrowed at a shoulder portion 51 adjacent to a proximal end of the channel 28 to prevent advancement and stop the biopsy cannula 31 and stylet 43 from exiting out a proximal end of the body portion 37. Likewise, the hub 45 that is fixed to the proximal end of the stylet 43 is sized larger than the narrowed shoulder portion 51 to stop advancement of the stylet 43 in the channel 28. A portion 28a of the channel 28 extending from the narrowed shoulder portion 51 to the proximal end of the body portion 37 is sized for receiving the guide wire 122, when used with the medical apparatus 29. Likewise, a similarly sized opening 53 is made in the palm band 50 in a coaxial relationship with the channel 28 such that the guide wire 122 can protrude through and exit the proximal end of the medical apparatus 29 when sliding the medical apparatus 29 over the guide wire 122.

Referring briefly back to FIG. 2 and to FIGS. 6-8, the body portion 37, hub 45, and biopsy cannula 31 having cooperating features to prevent relative rotation between these components during use. The biopsy cannula 31 includes a plurality of grooves 64 extending longitudinally along the biopsy cannula 31 from the proximal end to a position adjacent the recess 30, and disposed at a ninety degree angle from the flats 66. The body portion 37 includes a plurality of keys 68 extending into the channel 28 for engaging the proximal end of the biopsy cannula 31 to block insertion of the biopsy cannula 31 and for allowing insertion of the biopsy cannula 31 as the keys 68 are aligned with the grooves 64, and to prevent rotation of the biopsy cannula 31 as it is used. The hub 45 includes a pair of grooves 55 for similarly aligning with the keys 68 when inserting the stylet 43 into the channel 28.

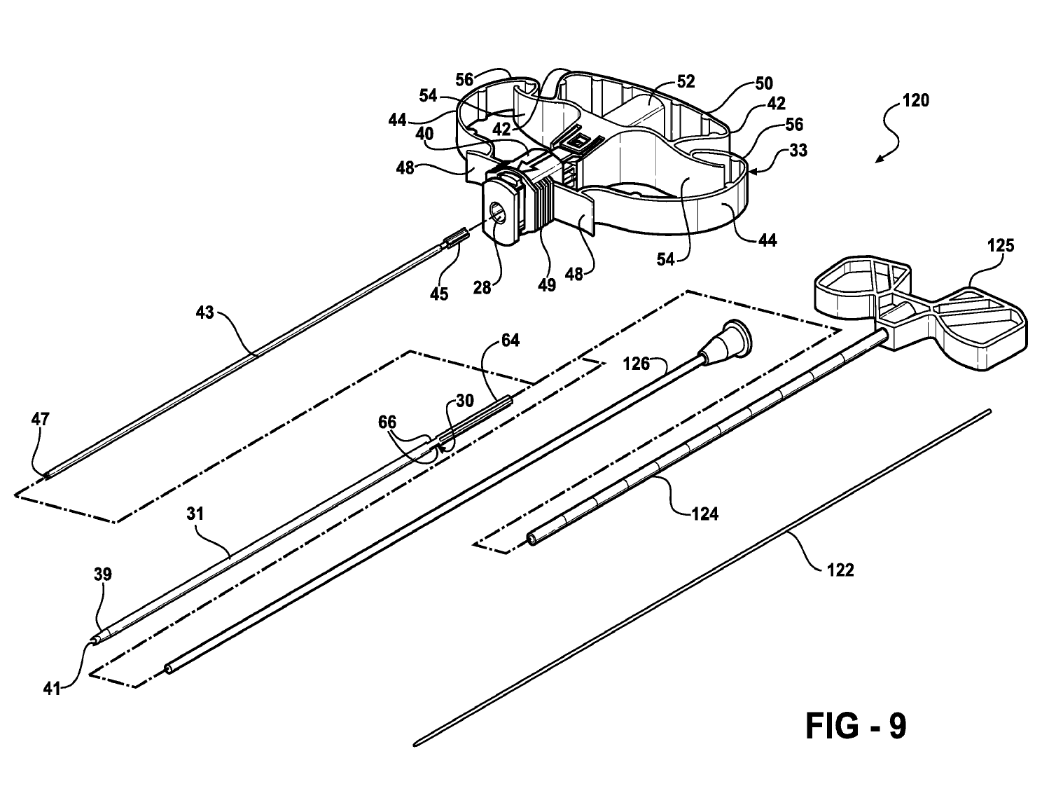

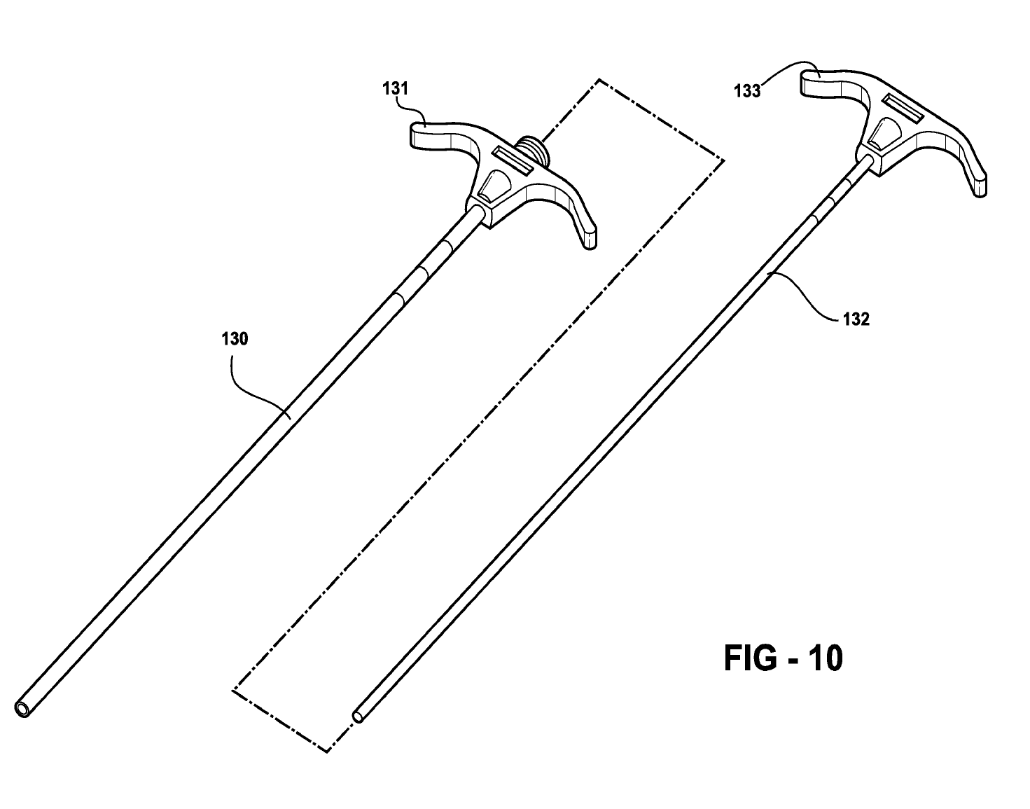

Referring to FIG. 9, the handle assembly 33, biopsy cannula 31, and stylet 43 may form part of a vertebral access and bone biopsy kit 120. Preferably, the biopsy cannula 31 with stylet 43 inserted therein is packaged while connected to the handle assembly 33 and shipped in this condition to reduce assembly and increase the ease of use for the ultimate end user. The kit may also include the guide wire 122, an access cannula 124, and an obturator 126. Referring to FIG. 10, cement delivery tools such as a cement cannula 130 and cement obturator 132 may be used with the biopsy kit 120 of FIG. 9 to form another kit. The components of these kits may be packaged in trays, envelopes, or other suitable packages and sterilized using conventional sterilization techniques. The following is a table of exemplary sizes and materials of the components of these kits. Of course, other sizes of these components within the scope of this invention could also be contemplated:

| | Outside Diam. | Inside Diam. | |
|---|---|---|---|
| Component | (in.) | (in.) | Material |
| guide wire 122 | 0.04 to 0.05 | — | 304 stainless steel |
| stylet 43 | 0.08 to 0.09 | 0.04 to 0.06 | 304 stainless steel |
| biopsy cannula 31 | 0.13 to 0.15 | 0.08 to 0.10 | 304 stainless steel |
| obturator 126 | 0.08 to 0.09 | — | 304 stainless steel |
| access cannula | 0.16 to 0.17 | 0.14 to 0.15 | 304 stainless steel |
| cement cannula 130 | 0.13 to 0.15 | 0.11 to 0.12 | 304 stainless steel |
| cement obturator | 0.10 to 0.12 | — | 304 stainless steel |

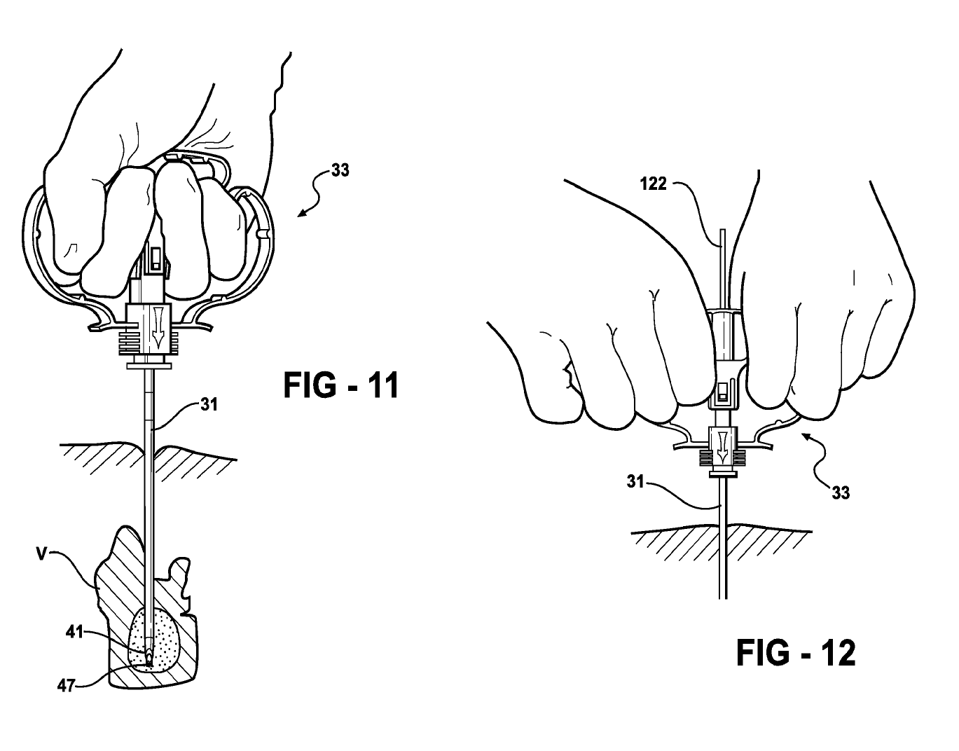

An exemplary procedure for taking a biopsy sample and injecting bone cement into the remote anatomical site using the biopsy kit 120 and cement delivery tools 130 and 132 is described and illustrated in FIGS. 11 through 18.

In a first step shown in either FIG. 11 (with the guide wire 122) or FIG. 12 (without the guide wire 122), the user first penetrates the tissue with the medical apparatus 29. In FIG. 11, the distal ends of the stylet 43 and the biopsy cannula 31 penetrate the tissue until the distal ends enter the remote anatomical site. In one embodiment, the medical apparatus 29 penetrates an interior of a vertebral body V (illustrated in FIG. 11) such as a vertebral body that has been compressed, e.g., vertebral compression fracture, due to trauma, osteoporosis, or other disease condition. In this instance, the biopsy kit 120 and cement delivery tools 130 and 132 are used to access cancellous bone tissue inside the vertebral body to obtain a biopsy specimen of the tissue and to repair the compression fracture by injecting PMMA bone cement or other material capable of setting to a hardened condition. The biopsy cannula 31 and stylet 43 are preferably advanced under fluoroscopic guidance through a pedicle of the vertebral body V. Twisting or hammering may be employed to facilitate penetration of bone.

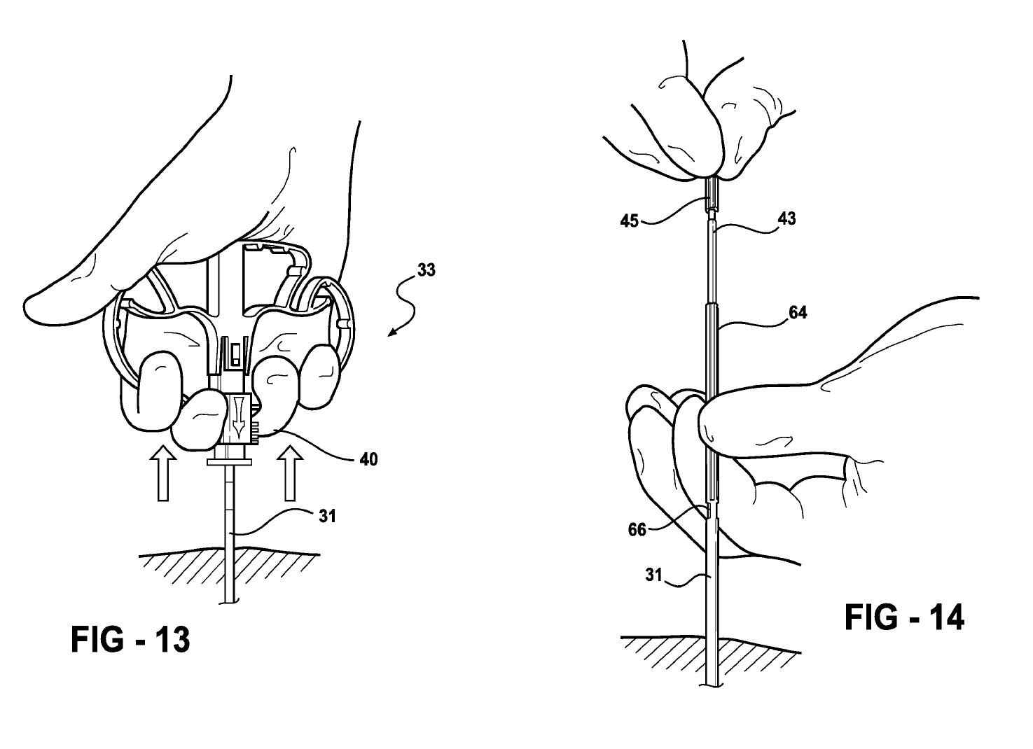

In a second step shown in FIG. 13, once the medical apparatus 29 has penetrated the tissue to the desired location, e.g., once the vertebral body has been entered, then the handle assembly 33 is released from the biopsy cannula 31 by sliding the collar 40 to the release position. The handle assembly 33 is then removed by holding the collar 40 in the release position and sliding the handle assembly 33 off of the biopsy cannula 31.

In a third step shown in FIG. 14, the stylet 43 is removed by grasping the hub 45 and pulling the stylet 43 straight out from a bore of the biopsy cannula 31.

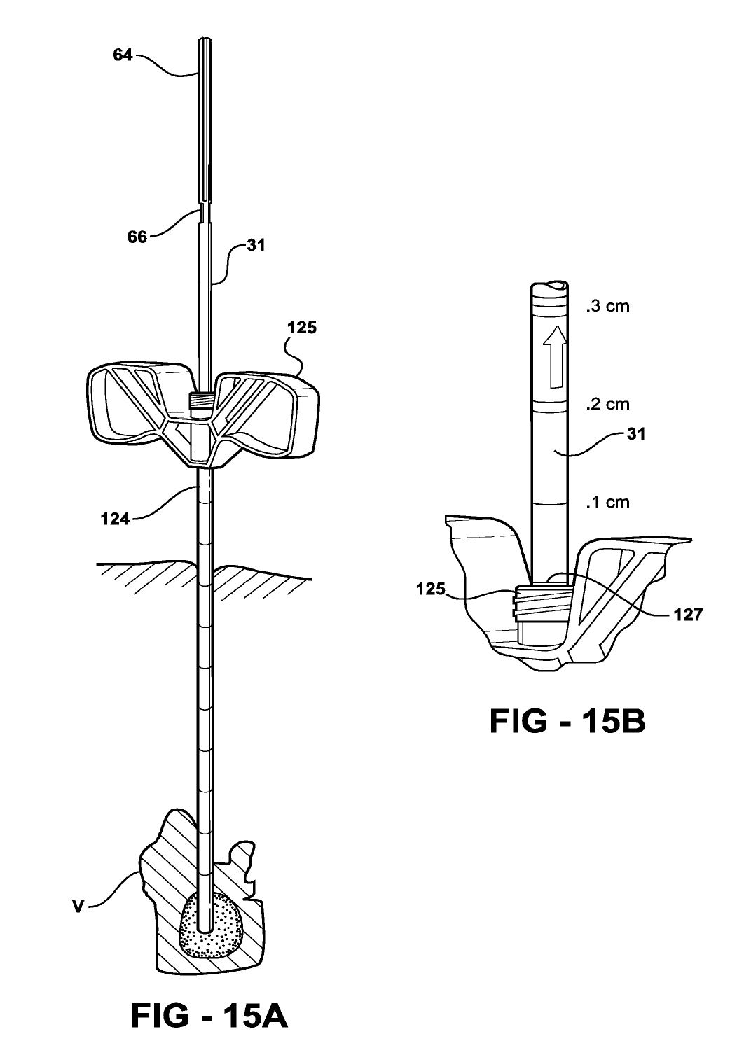

In a fourth step shown in FIGS. 15A and 15B, the access cannula 124 is advanced over the biopsy cannula 31 until the access cannula 124 penetrates the vertebral body V generally to the depth that the biopsy cannula 31 has penetrated the vertebral body V. The access cannula 124 is also advanced under fluoroscopic guidance. Referring to FIG. 15B, the access cannula 124 is slid over the biopsy cannula 31 until a mark 127 on the biopsy cannula 31 is aligned with a handle 125 fixed to a proximal end of the access cannula 124. The access cannula 124 is sized for sliding over the biopsy cannula 31 and the recess 30 with minimal spacing between an outer surface of the biopsy cannula 31 and an inner surface of the access cannula 124 to provide a slip-fit. Again, twisting or hammering may be employed to advance the access cannula 124 through bone tissue.

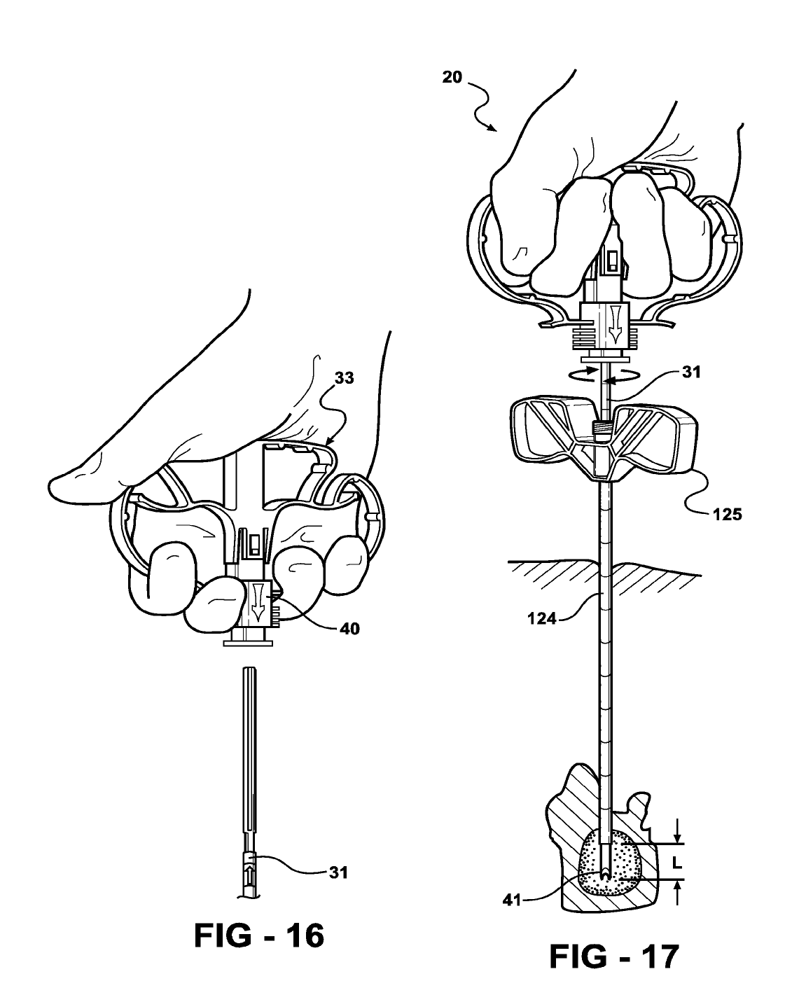

In a fifth step shown in FIG. 16, the handle assembly 33 is once again placed on the biopsy cannula 31. The user slides the collar 40 to the release position and then places the handle assembly 33 over the biopsy cannula 31, i.e., by sliding the biopsy cannula 31 into the channel 28. Once the catches 36 are in position in the recess 30, the collar 40 is released to the lock position to hold the catches 36 in the recess 30. An arrow marked on the collar 40 such as by printing or embossing is to be aligned with an arrow marked on the biopsy cannula 31 to facilitate mounting the handle assembly 33 to the biopsy cannula 31.

In a sixth step shown in FIG. 17, once the handle assembly 33 is firmly secured to the biopsy cannula 31, then a biopsy specimen is taken by further advancing the biopsy cannula 31 into the vertebral body V. Depth markings on the biopsy cannula 31 are used to determine the depth of penetration and the associated length L of the biopsy specimen. Once the biopsy specimen is obtained, rotate the handle assembly 33 and biopsy cannula 31 at least one half turn to score the biopsy specimen to facilitate its removal. Twisting and pulling motions on the handle assembly 33 are then used to remove the biopsy cannula 31 from the access cannula 124 to ensure that the biopsy specimen is retained in the biopsy cannula 31. The obturator 126 can then be used to remove the biopsy specimen from the biopsy cannula 31 in a known manner. At this point in the procedure, the access cannula 124 remains in the vertebral body V to provide access to the vertebral body V to deliver PMMA bone cement, medications, and/or other materials.

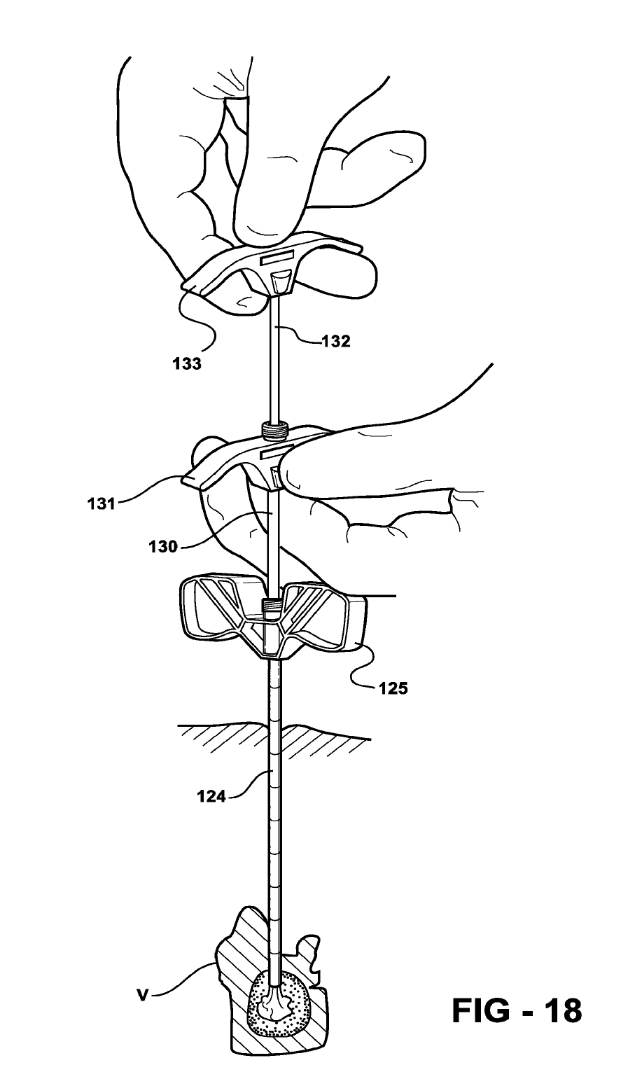

In a final step shown in FIG. 18, the cement cannula 130 and cement obturator 132 are used to deliver PMMA bone cement to the vertebral body through the access cannula 124. The cement cannula 130 is preferably a tubular body with a corresponding handle 131 fixed to a proximal end of the tubular body. The cement obturator 132 is preferable a solid rod with a corresponding handle 133 fixed to a proximal end of the solid rod. The solid rod is sized to slidably fit into the tubular body of the cement cannula 130 to force materials loaded into the tubular body therefrom. In FIG. 18, the cement cannula 130 is first loaded with PMMA bone cement and then inserted into the access cannula 124 until a distal end of the cement cannula 130 is positioned at a desired delivery location when viewed under fluoroscopy. Next, the cement obturator 132 is inserted into the cement cannula 130 to inject the PMMA bone cement into the vertebral body V. When complete, the cement cannula 130 and cement obturator 132 are removed from the access cannula 124 and the access cannula is removed from the patient. The handles 125, 131, 133 fixed

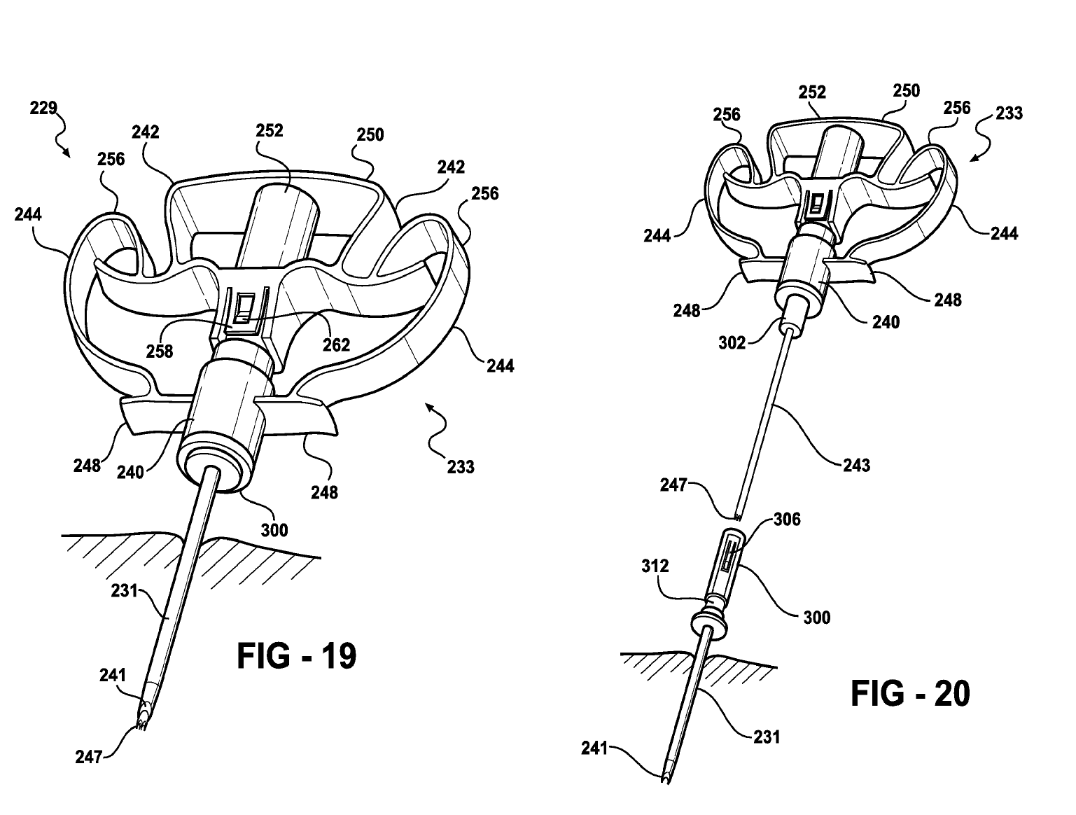

An alternative embodiment of the medical apparatus 229 and method of use is described with reference to FIGS. 19-27. In this embodiment, the handle assembly 233 is as previously described, but having a generally larger channel 228 for receiving the biopsy cannula 231, the stylet 243, and the access cannula 224. Furthermore, in this embodiment, each of the biopsy cannula 231, stylet 243, and access cannula 224 are outfitted with a hub 300, 302, 304 for interfacing with the handle assembly 233. The biopsy cannula 231, stylet 243, and access cannula 224, are coupled to their respective hubs 300, 302, 304, and the hubs 300, 302, 304 are inserted into the channel 228 of the body portion 237 of the handle assembly 233, permitting these medical devices to be removably coupled to the handle assembly 233.

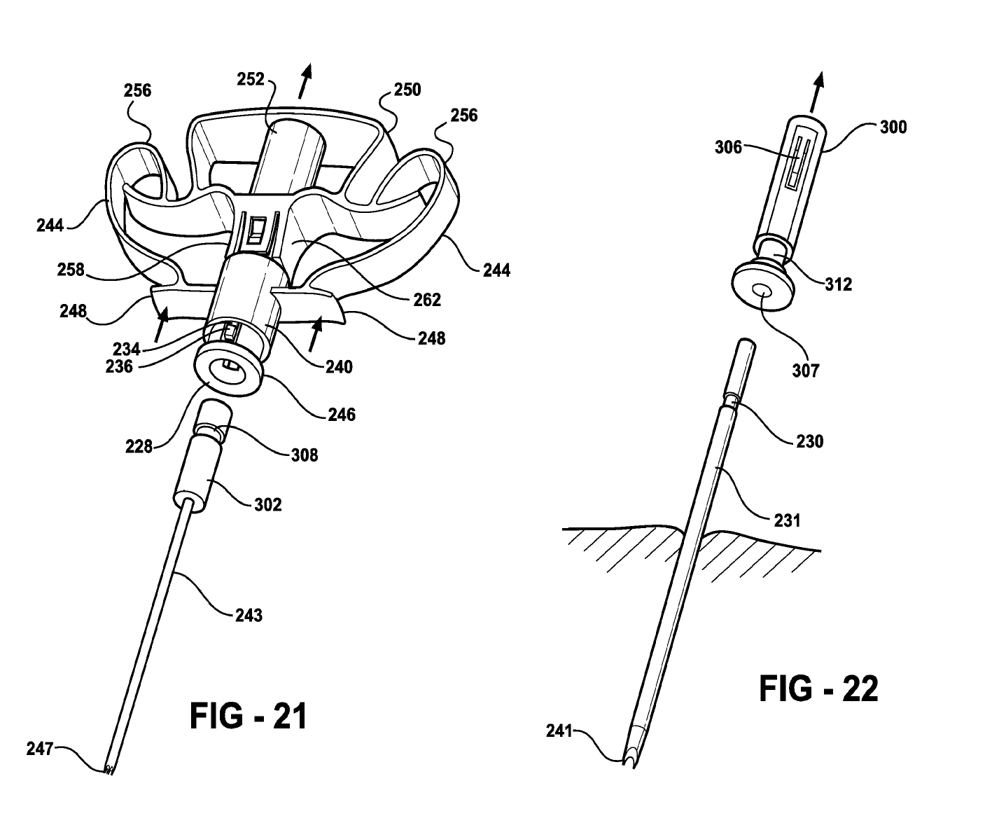

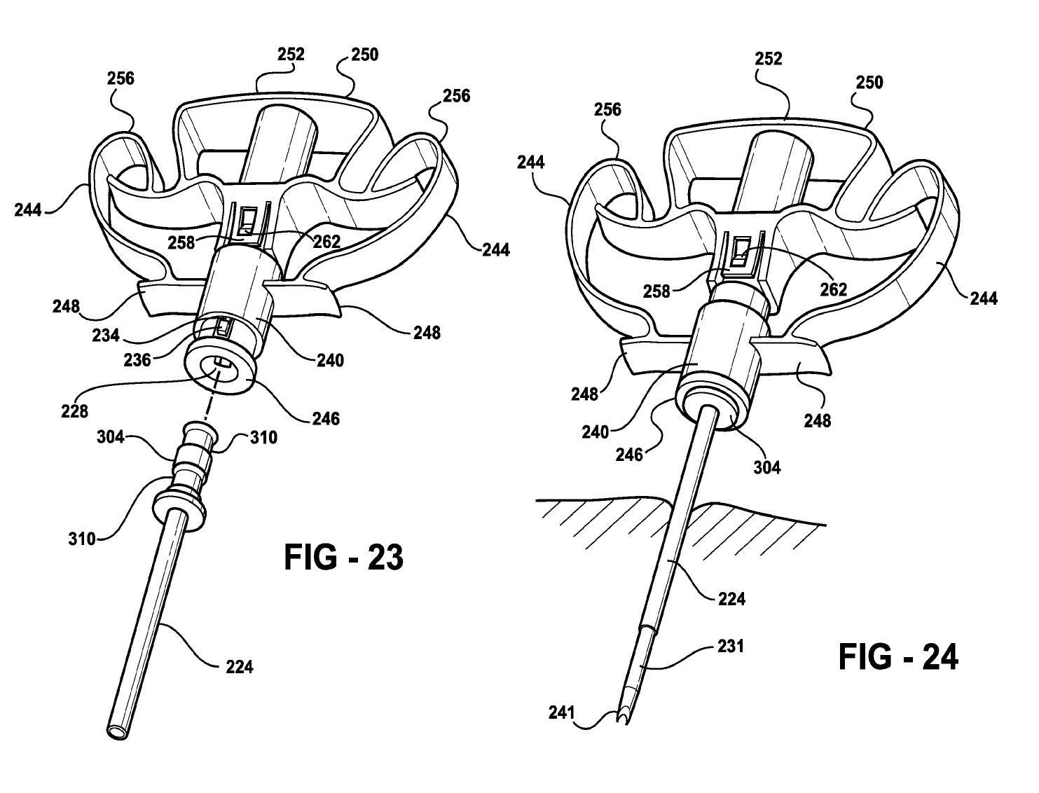

The stylet 302 and access cannula 304 hubs may be fixed to a proximal end of the stylet 243 and access cannula 224, respectively, such as by adhesive, UV curable material, welding, or the like. FIG. 21 illustrates the stylet 243 with associated stylet hub 302 fixed thereto. The stylet hub 302 includes a recess 308 for receiving the catches 236 of the detent arms 234 of the handle assembly 233 as previously described. FIG. 23 illustrates the access cannula 224 with associated hub 304 fixed thereto. The access cannula hub 304 also includes a pair of recesses 310 for receiving the catches 236 of the detent arms 234 at varying axial positions along the access cannula 224.

Referring to FIG. 22, the hub 300 for the biopsy cannula 231 is shown. The biopsy cannula hub 300 is preferably releasably coupled to the biopsy cannula 231. More specifically, the biopsy cannula hub 300 defines a bore 307 for receiving the proximal end of the biopsy cannula 231. A pair of opposing detent arms 306, similar to the detent arms 34 of the body portion 37, are formed in cut-out portions of the biopsy cannula hub 300. The detent arms 306 lock in the recess 230 defined in the biopsy cannula 231 in precisely the same manner as the detent arms 34 lock in the recess 30 in the preferred embodiment. When the biopsy cannula 231 with associated biopsy cannula hub 300 coupled thereto is positioned in the channel 228, the detent arms 306 are restrained from outward radial movement, thereby locking the biopsy cannula hub 300 to the biopsy cannula 231. The biopsy cannula hub 300 further includes a separate recess 312 for receiving the catches 236 of the detent arms 234 in the handle assembly 233. Notably, in this embodiment, the stylet 243 and access cannula 224, in addition to the biopsy cannula 231, can be manipulated using the handle assembly 233, thereby providing a universal handle assembly 233.

Operation of this alternative medical apparatus 229 is illustrated in a series of steps shown in sequence in FIGS. 19-27.

In FIG. 19, the medical apparatus 229 is first inserted into the patient to access the remote anatomical site. The site may be a vertebral body, or other anatomical site, which is not specifically shown for convenience.

In FIG. 20, once the medical apparatus 229 is inserted to a desired depth, the handle assembly 233 is released from the biopsy cannula hub 300. The handle assembly 233 is then used to grasp the stylet hub 302 to remove the stylet 243 from the biopsy cannula 231.

In FIG. 21, the stylet hub 302 is released from the handle assembly 233.

In FIG. 22, the biopsy cannula hub 300 is removed from the biopsy cannula 231 to leave the biopsy cannula 231 in place in the patient.

In FIG. 23, the access cannula hub 304 is inserted into the handle assembly 233 to couple the access cannula 224 to the handle assembly 233.

In FIG. 24, the access cannula 224 is then slid over the biopsy cannula 231 (without biopsy cannula hub 300) until the distal end of the access cannula 224 is approximately aligned axially with the distal end of the biopsy cannula 231.

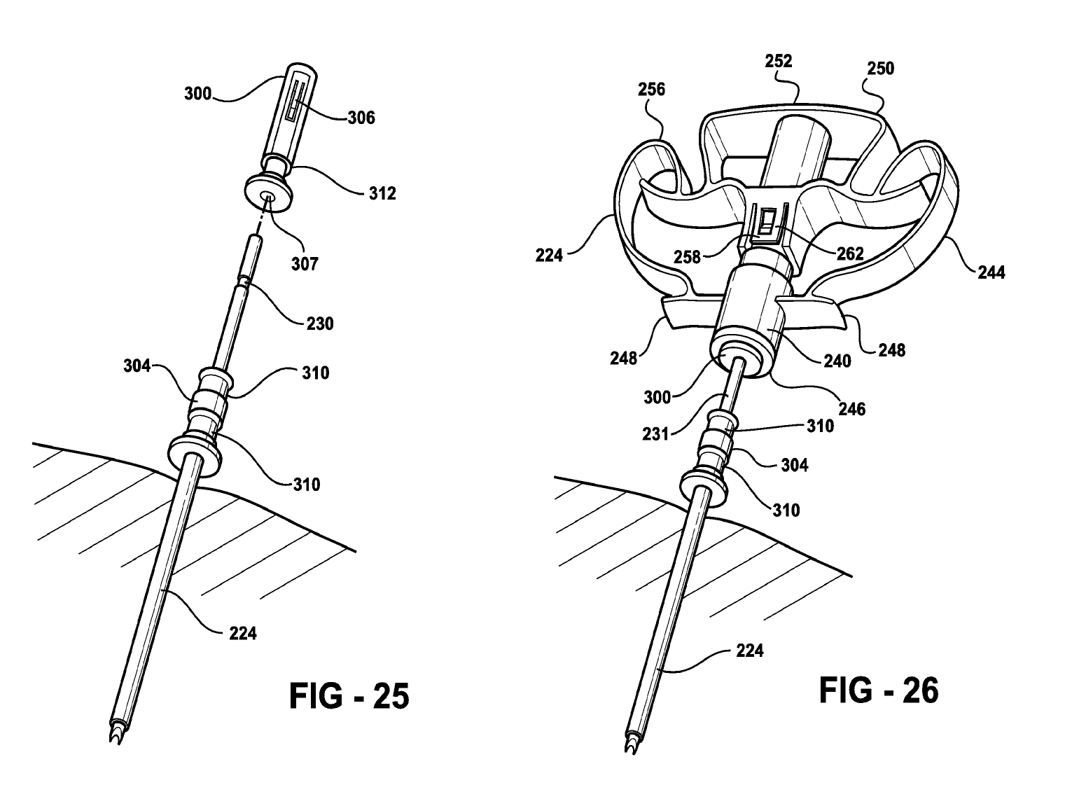

In FIG. 25, the handle assembly 233 has been removed from the access cannula hub 304 and the biopsy cannula hub 300 is repositioned on the biopsy cannula 231.

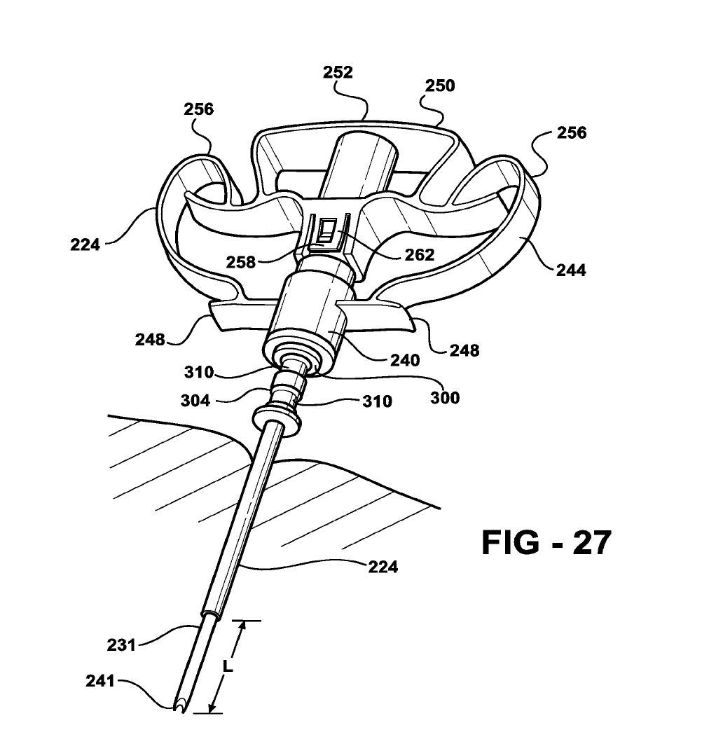

In FIG. 26, the handle assembly 233 grasps the biopsy cannula hub 300 to re-couple the biopsy cannula 231 to the handle assembly 233.

In FIG. 27, the biopsy specimen is then obtained by advancing the biopsy cannula 231 distally in accordance with the principles described above in the preferred embodiment. It should be appreciated that the hubs 300, 302, 304 may include grooves and the channel 228 may include keys, similar to the grooves and keys described above in the preferred embodiment, to prevent relative rotation between the handle assembly 233 and the biopsy cannula 231, stylet 243, and/or access cannula 224. The bore 307 in the biopsy cannula hub 300 may likewise include keys with corresponding grooves formed on a proximal end of the biopsy cannula 231 to prevent relative rotation between the biopsy cannula hub 300 and the biopsy cannula 231.

Obviously, other modifications and variations of the present invention are possible in light of the above teachings. The invention may be practiced otherwise than as specifically described within the scope of the appended claims. The reference numerals in the claims are merely for convenience and are not to be read in any way as limiting.

What is claimed is:

1. A medical apparatus for penetrating through tissue to access a remote anatomical site, comprising:
a cannula (31) having a proximal end and a distal end shaped for accessing the anatomical site, said cannula (31) having a locking feature (30) adjacent said proximal end and an outer diameter of 8 gauge or smaller; and
a handle assembly (33) including;
a handle portion (35) for manually grasping said handle assembly (33),
a body portion (37) supported by said handle portion (35) and defining a channel (28) for receiving said proximal end of said cannula (31),
said body portion (37) presenting a detent (32) moveable between an inward position extending into said channel (28) for engaging said locking feature (30) to retain said cannula (31) and an outward position for disengaging from said locking feature (30) to allow said cannula (31) to move in and out of said body portion (37), and
a component movable relative to said body portion (37) between a lock position for holding said detent (32) in said inward position and a release position for allowing said detent (32) to move to said outward position.

2. An apparatus as set forth in claim 1 wherein said detent (32) is normally biased to said inward position.

3. An apparatus as set forth in claim 2 wherein said detent (32) comprises a pair of detent arms (34) integral with said body portion (37) and extending to distal ends and a catch (36) extending laterally from each of said distal ends for engaging and retaining said cannula (31).

4. An apparatus as set forth in claim 3 including a spring to urge and hold said component in said lock position and to allow said component to be moved along said body portion (37) to said release position.

5. An apparatus as set forth in claim 4 wherein said body portion (37) presents a stop (46) for limiting movement of said component to establish said lock position by reacting against said spring.

6. An apparatus as set forth in claim 5 including at least one grip tab (48) extending from said component for manually moving said component along said body portion (37).

7. An apparatus as set forth in claim 6 wherein said component comprises a collar (40) surrounding said body portion (37), and said handle portion (35) includes a grasping portion (42) for manually grasping and supporting said apparatus, and said spring includes a pair of flexible arms (44) extending in a bendable path to interconnect said grasping portion (42) and said collar (40) for urging said collar (40) to said lock position and for bending in response to a force to allow said collar (40) to be moved along said body portion (37) to said release position.

8. An apparatus as set forth in claim 7 including a pair of said grip tabs (48) extending radially in opposite directions from said collar (40), and wherein said flexible arms (44) extend in opposite directions from said grip tabs (48) in outwardly bowed sections.

9. An apparatus as set forth in claim 8 wherein said grasping portion (42) includes a curved palm band (50) and center post (52) extending from said palm band (50) and supporting said body portion (37) and a pair of finger grips (54) extending radially from said post (52).

10. An apparatus as set forth in claim 9 including a mechanical connection interconnecting said body portion (37) and said post (52).

11. An apparatus as set forth in claim 10 wherein said mechanical connection includes a pair of axially extending and flexible flaps (58) presented by said post (52) and defining apertures (60) therein and lugs (62) extending radially from said body portion (37) for disposing in said apertures (60) to retain said body portion (37) to said post (52).

12. An apparatus as set forth in claim 3 wherein said channel (28) is defined by a bore extending axially into said body portion (37) and said body portion (37) defines side openings (38) extending radially into said bore and said detent arms (34) are disposed in said openings (38).

13. An apparatus as set forth in claim 12 wherein said handle portion (35) and said flexible arms (44) and said collar (40) comprise an integral polymeric material.

14. An apparatus as set forth in claim 13 wherein said body portion (37) comprises said organic material.

15. An apparatus as set forth in claim 3 wherein said cannula (31) defines a recess (30) adjacent one end thereof for receiving said catches (36) to retain said cannula (31) in said body portion (37).

16. An apparatus as set forth in claim 15 wherein said recess (30) comprises a pair of flats (66) on opposite sides of said cannula (31).

17. An apparatus as set forth in claim 1 wherein said cannula (31) defines a plurality of grooves (64) extending longitudinally along at least a portion of said cannula (31).

18. An apparatus as set forth in claim 17 wherein said body portion (37) includes a pair of keys (68) extending into said channel (28) for engaging said proximal end of said cannula (31) to block insertion of said cannula (31) and for allowing insertion of said cannula (31) as said keys (68) are aligned with said grooves (64).

19. An apparatus as set forth in claim 18 including a stylet (43) having a proximal end and a distal end, said stylet (43) including a hub (45) adjacent said proximal end wherein said hub (45) includes grooves for mating with said keys (68) in said body portion (37) to allow said stylet (43) to be inserted with said cannula (31) in said handle assembly (33).

20. A medical apparatus for penetrating through tissue to access a remote anatomical site, comprising:
a first cannula (31) having a proximal end and a distal end shaped for accessing the anatomical site, said first cannula (31) having a first locking feature (30) adjacent said proximal end;
a second cannula (124) slidable over said first cannula (31) and said first locking feature (30); and
a handle assembly (33) including;
a handle portion (35) for manually grasping said handle assembly (33),
a body portion (37) supported by said handle portion (35) for receiving said proximal end of said first cannula (31), said body portion (37) presenting a second locking feature (32) moveable between a first position for engaging said first locking feature (30) to retain said cannula (31) and a second position for disengaging from said first locking feature (30) to allow said first cannula (31) to move in and out of said body portion (37), and
a component movable relative to said body portion (37) between a lock position for holding said second locking feature (32) in said first position and a release position for allowing said second locking feature (32) to move to said second position.
